(19) United States
(12) Patent Application Publication
(10) Pub. No.: US 2007/0196321 A1
(43) Pub. Date: Aug. 23, 2007

(54) In vivo stimulation of intestinal transporters for excretion of nitrogenous wastes

(76) Inventor: Alan D. Strickland, Lake Jackson, TX (US)

Correspondence Address: BELL, BOYD & LLOYD LLP, P.O. BOX 1135, CHICAGO, IL, 60690, US

(21) Appl. No.: 11/549,514

(22) Filed: Oct. 13, 2006

(30) Foreign Application Priority Data
Apr. 12, 2005 (US) PCT/US05/12237

(51) Int. Cl. A61K 31/74

(52) U.S. Cl. 424/78.01

(57) ABSTRACT

A method for stimulating active transporters of metabolic waste, in particular urea and creatinine, in the GI tract of a mammal, comprising the step of administering an effective amount of a concentrator activation agent to the intestinal tract of the mammal, is disclosed. Methods for concentrating metabolic wastes in the intestinal tract to be above those achieved through passive diffusion alone, are also disclosed.

### FILED OF THE INVENTION

The present invention relates to a method for improving the excretion of metabolic wastes, particularly urea and creatinine.

### BACKGROUND OF THE INVENTION

Metabolism of food substances produces waste products. Major waste products from the metabolism of proteins are nitrogenous substances such as urea, creatinine, and uric acid or urates. Water is also formed in large quantities during metabolic breakdown of foods. Several minerals, such as potassium, sodium, and phosphate are released during the metabolic process. In general, these water-soluble waste products and the water produced during metabolism are excreted via the urinary system.

In the past, it was understood that the glomerulus filtered all molecules below a certain size including both nutrients and wastes so that these small molecules entered the renal tubular system for excretion as urine. The proximal renal tubules were known to have active transport processes to reabsorb nutrients such as glucose, sodium, water, calcium, phosphate, hydrogen, and amino acids. (see Renal Physiology, Third Edition, Bruce M. Koeppen and Bruce A. Stanton, Mosby, St. Louis, 2001, pp 31-167 and Principles of Renal Physiology, Fourth Edition, Christopher J. Lote, Kluwer, London, 2000, pp 34-165.). This process is quite efficient with 100% of the glucose and amino acids being reabsorbed, 70% of the filtered water being reabsorbed, and 68% of the filtered sodium being reabsorbed in the proximal tubule. Nitrogenous waste products passively follow water movement (see Lote, pp 164-165) in the proximal tubule, although only about 50% of the filtered urea is reabsorbed in the proximal tubule (see Lote, p 76-78). The tubular fluid is isotonic with plasma throughout the passage from glomerular filtration to the end of the proximal tubule. After the tubular fluid passes from the proximal tubule into the cortical renal tubules, the main task is to concentrate the urine so that the correct amount of electrolytes and water will be excreted to maintain the body homeostasis. It was understood that various portions of the cortical and medullary renal tubule allowed different substances to pass at different rates due to differential membrane permeability to the different substances. The descending limb of the loop of Henle was understood to have epithelial cells which freely allowed water and urea to move through the cells but were only partially permeable to sodium (see Lote, pp 70-85). The ascending limb of the loop of Henle was understood to have epithelial cells that were impermeable to water and urea while actively pumping sodium out of the tubular lumen into the renal interstitium. This lowered the concentration of sodium in the renal tubule while urea concentration increased dramatically. Through a countercurrent multiplication arrangement, this resulted in a marked increase in solute concentration in both the tubule and the interstitium in the renal medulla. Another 20% of the filtered fluid volume and 20% of the filtered sodium was reabsorbed during the movement through the loop of Henle. None of the urea was reabsorbed in this passage. When the tubular fluid left the loop of Henle and entered the cortical distal tubule, impermeability of the epithelial membrane to urea continued to result in increasing concentrations of urea while sodium was actively pumped out of the tubule resulting in hypo-osmolar fluid. The membrane of the medullary collecting duct was understood to be permeable to urea, resulting in diffusion of urea out of the tubular fluid and into the medullary interstitial space. This causes a very high concentration of urea in the medullary interstitium so that urea passively diffuses into the proximal, descending limb of the loop of Henle and as much as 50% of the high interstitial osmolarity of the medullary tissue is due to urea. As the tubular fluid passes through the medullary collecting duct, the interstitial hyperosmolarity results in final concentration of the urine. When it was discovered that the permeability of the collecting duct to urea changed with varying levels of antidiuretic hormone (ADH), it was decided that the transport of urea in this site was not simple diffusion across the lipid bilayer of the epithelial cells but was facilitated diffusion through a pore or a uniporter that opened or closed in response to ADH. (See Lote p 78 and Koeppen p82)

The gastrointestinal tract has also been examined for movement of water, nutrients, and waste products such as urea. Initial studies indicated that urea moved passively in either direction between the bloodstream and the intestinal lumen depending on concentration (“The passage of urea between the blood and the lumen of the small intestine.” Pendleton, W. R. and West, F. E. Am. J. Physiol. 1932; 101: 391-395). Later, studies were performed in regards to urea utilization in the gastrointestinal tract due to a desire to inexpensively feed ruminants diets higher in nitrogen than typical straw diets without having to use expensive grains with higher protein contents than straw. One source of the nitrogen investigated was urea (“Urea transport in gastrointestinal tract of ruminants: effect of dietary nitrogen.” Ritzhaupt, A., Breves, G., Schroder, B., Winckler, D., And Shirazi-Beechey, S. Bioch Soc Transact. 1997; 25: 490S. “Transport of urea nitrogen from the intestines into the stomach in dairy cows.” Voigt, J. and Piatkowski, B. Archiv fur Tierernahrung 1984; 34: 769-784.). The studies sought to determine the movement of unchanged urea versus the possible conversion of urea to amino acids by bacteria in the ruminants' stomachs and subsequent absorption of the amino acids. Since there is also high bacterial colonization of the colon (large intestine), the possibility of production of amino acids by colonic bacteria followed by absorption of those amino acids was also examined. Small intestinal studies were performed as well. Studies were extended to non-ruminants such as dogs. The conclusions were that urea was useful for adding nitrogen to the feed of ruminants, but that absorption of intact urea was not important. Authors reported that the intact small intestinal mucosa moved urea in either direction (absorption or secretion) only by passive diffusion governed by sieving coefficients that made the movement of urea 10 times less than that of the water it was passively following (see “Urea movement trough intestinal epithelium,” Lifson, N. Urea, Kidney, Proc. Int. Colloquy. 1970; 114-118. “Contribution of solvent drag to the intestinal absorption of tritiated water and urea from the jejunum of the rat.” Ochsenfahrt, H. and Winne, D. Naunyn-Schmiedeberg Archives of Pharmacology. 1973; 279: 133-152. “Vascular flow of the compartmental distribution of transported solutes within the small intestinal wall.” Boyd, C. International Congress Series 1977; 391 (Intestinal Permeation): 41-47. “Influence of anesthetic regimens on intestinal absorption in rats.” Yuasa, H., Matsuda, K., Watanabe, J. Pharma Res 1993; 10: 884-888.). Studies in humans agreed with the passive movement of urea in the small intestine so that urea was suggested as a good hyperosmotic agent for studies of water and solute movement in the intestine that would not itself significantly move while the movements of the other compounds were occurring (“Stimulation of active and passive sodium absorption by sugars in the human jejunum.” Fordtran, J. J Clin Invest 1975; 55: 728-737. “Mechanism of isoosmotic transport of fluid across the small intestine. Effect of the Staverman reflection coefficient of the solute used to increase the osmolality of the mucosal solution on the composition of the absorbate.” Beck, I. and Dinda, P. Canadian J Physiol and Pharm. 1974; 52: 96-104. “Effect of D-glucose on intestinal permeability and its passive absorption in human small intestine in vivo.” Fine, K., Santa Ana, C., Porter, J., Fordtran, J. Gastroenterology 1993; 105: 1117-1125.). Similarly, urea movement into and out of the colon was understood to be passive with a low permeability (“Transfer of blood urea into the goat colon.” Von Engelhardt, W and Hinderer, S. Tracer Stud Non-Protein Nitrogen Ruminants 3, Proc Res Co-Ord Meet. 1976; 57-58. “Ammonia and urea transport by the excluded human colon.” Brown, R., Gibson, J., Fenton, J., Snedden, W., Clark, M., and Sladen, G. Clin Sci Molec Med 1975; 48: 279-287. “The effects of intravenous urea infusions in the portal and arterial plasma ammonia and urea enrichment of jejunal and colonic infusions.” Malmloef, K. and Nunes, C. Scand J Gastro 1992; 27: 620-624.). The understanding was that the permeability to passive diffusion was determined by paracellular pores which could be damaged causing increased leakage of urea (“Comparative assessment of intestinal transport of hydrophilic drugs between small intestine and large intestine.” Yuasa, H., Matsuda, K., Kimura, Y., Soga, N., and Watanabe, J. Drug Delivery 1997; 4: 269-272. “Entry of blood urea into the rumen of the llama.” Hinderer, S. and Von Engelhardt. Tracer Stud Non-Protein Nitrogen Ruminants 3, Proc Res Co-Ord Meet. 1976; 59-60. “Jejunal dialysis. I. The effect in the dog of local iodoacetate on the dialysis of urea, creatinine, inorganic phosphorus, and xylose.” Meyer, R., Cohen, W. Solis, J, and LeBeau, R. Metabolism, Clinical and Experimental 1962; 11, 999-1014.).

In recent studies of the renal mechanisms for movement of solutes and water in the kidney, transporters have been found and described for three of the nitrogenous waste compounds. A sodium-coupled transporter of creatine has been described in neurological tissue, but the title of the article in literature searches is erroneously reported to concern creatine (“Family of sodium-coupled transporters for GABA, glycine, praline, betaine, taurine, and creatinine: pharmacology, physiology, and regulation.” Deken, S., Fremeau, R., and Quick, M. Neurotransmitter Transporters, Second Edition, Humana Press, Totowa, N.J. 2002: 193-233.). The true title of the actual article has the word creatine and deals with movement of the neurologically active compound creatine. No literature reports of transporters for creatinine in renal tissue or any other tissue have been found. A urate transporter (URAT1 encoded by Slc22a12) has been described in the renal tubule (“Urate transporter and renal hypouricemia.” Enomoto, Atsushi; Niwa, Thosimitsu; Kanai, Yoshikatsu; Endou, Hitoshi. Rinsho Byori 2003; 51(9): 892-897, “Function and localization of urate transporter I in mouse kidney.” Hosoyamada, Makoto; Ichida, Kimiyoshi; Enomoto, Atsushi; Hosoya, Tatsuo; Endou, Hitoshi. J. Am. Soc. Neph 2004; 15(2), 261-268, and “Mechanism of urate transport in the human kidney.” Enomoto, Atsushi. Jin to Toseki 2003; 55(2), 264-269). These transporters reclaim urates from the tubular lumen for use in the bloodstream as antioxidants. Mutations in Slc22a12 have been found in patients with gout. No literature reports investigate the possibility of urate transporters in the intestinal tract.

A family of urea transporters have recently been discovered. Five isoforms of UT-A (urea transporter A) and two isoforms of UT-B (urea transporter B) have been described. The UT-A transporters are all transcribed from a set of 24 exons via the action of two promoters, one of which is vasopressin sensitive (“Cloning of the rat Slc14a2 gene and genomic organization of the UT-A urea transporter.” Nakayama, Y.; Naruse, M.; Karakashian, A.; Peng, T.; Sands, J. M.; Bagnasco, S. M. Biochimica et Biophysica Acta 2001; 1518(1-2): 19-26). This allows variable expression of each isoform of UT-A in different tissues or portions of tissues and also allows expression of the protein in a tissue even though the protein is not active in that tissue. All of the UT-A isoforms are facilitated diffusion urea transporters (“Regulation of renal urea transporters.” Sands, J. J. Am. Soc. Nephrol. 1999; 10(3): 635-646.). UT-A1 is a vasopressin-sensitive, glucocorticoid-regulated isoform found in the apical membrane of distal renal medullary collecting duct cells, as well as the inner ear, the heart, and liver (“Glucocorticoids inhibit transcription and expression of the UT-A urea transporter gene.” Peng, Tao; Sands, Jeff M.; Bagnasco, Serena M. Am J Physiology 2002; 282(5, Pt. 2): F853-858; “Immunohistochemical localization of urea transporters A and B in the rat cochlea.” Kwun, Yong-Sig, Yeo, Sang W., Ahn, Yang-Heui, Lim, Sun-Woo, Jung, Ju-Young, Kim, Wan-Young, Sands, Jeff M., Kim, Jin. Hearing Research 2003; 183(1-2): 84-96; “The Slc14 gene family of urea transporters.” Shayakul, C. and Hediger, M. Pfluegers Archiv. 2004; 447(5), 603-609; and “Mammalian urea transporters.” Sands, Jeff M. Annual Review of Physiology 2003; 65: 543-566). UT-A1 has been found to be active in the renal medullary collecting tubule and the inner ear, but no activity has been described in the heart or liver despite the expression in those tissues. UT-A2 is a facilitated transporter of urea located in both the proximal and distal medullary tubules (“Correction of age-related polyuria by dDAVP: Molecular analysis of aquaporins and urea transporters.” Combet, Sophie; Geffroy, Nancy; Berthonaud, Veronique; Dick, Bernhard; Teillet, Laurent; Verbavatz, Jean-Marc; Corman, Bruno; Trinh-Trang-Tan, Marie-Marcelle. Am J Physiology 2003; 284(1, Pt. 2): F199-F208). UT-A1 is described as a 117 kDa protein while UT-A2 is 97 kDa (“Aquaporin-2 and urea transporter-A-1 are up-regulated in rats with Type I diabetes mellitus.” Bardoux, P., Ahloulay, M., LeMaout, S., Bankir, L., and Trinh-Trang-Tan, M. Diabetologia 2001; 44(5): 637-546). UT-A3 is similar to UT-A1 in glucocorticoid regulation. UT-A3 and UT-A4 are active in the renal medullary collecting duct. UT-A5 is active in the testis but is not found in other tissues (“The Slc14 gene family of urea transporters.” Shayakul, C. and Hediger, M. Pfluegers Archiv. 2004; 447(5), 603-609.).

UT-B is encoded by the Slcl4a1 gene (“The Slc14 gene family of urea transporters.” Shayakul, C. and Hediger, M. Pfluegers Archiv. 2004; 447(5), 603-609.). The two isoforms of UT-B arise from differential utilization of two alternate polyadenylation signals (“Molecular characterization of a novel UT-A urea transporter isoform (UT-A5) in testis.” Fenton, R., Howorth, A., Cooper, G., Meccariello, R., Morris, I., Smith, C. Am. J. Physiol. Cell Physiol. 2000; 279: C1425-C1431). UT-B is a facilitated diffusion urea transporter found in many tissues, including the renal descending vasa recta, the inner ear, red blood cells, liver, colon, lung, testis, and brain (“Regulation of renal urea transporters.” Sands, J. J. Am. Soc. Nephrol. 1999; 10(3): 635-646, “Localization of the urea transporter UT-B protein in human and rat erythrocytes and tissues.” Timmer, R., Klein, J., Bagnasco, S., Doran, J., Verlander, J., Gunn, R., and Sands, J. Am. J. Physiol. 2001; 281(4, Pt 1), C1318-C1325.). UT-B activity has been demonstrated in the inner ear, the Sertoli cells of the testis, the vasa recta, and the erythrocyte membrane (“Immunohistochemical localization of urea transporters A and B in the rat cochlea.” Kwun, Y., Yeo, S., Ahn, Y., Lim, S., Jung, J., Kim, W., Sands, J., and Kim, J. Hearing Research 2003; 183(1-2): 84-96, “Coordinated expression of UT-A and UT-B urea transporters in rat testis.” Fenton, R., Cooper, G., Morris, I., and Smith, C. Am. J. Physiol. 2002; 282(6, Pt 1): C1492-C1501, “Lack of UT-B in vasa recta and red blood cells prevents urea-induced improvement of urinary concentrating ability.” Bankir, L., Chen, K., and Yang, B. Am. J. Physiol. 2004; 286 (1, Pt 2), F144-F151.). In the inner ear, urea is used to induce rapid changes in the volume and osmolality of the inner ear fluid. UT-B has been shown to be the Kidd blood group antigen (Jk) on red blood. Thus, the UT-A transporters in the collecting ducts move urea into the interstitial fluid of the renal medulla, the UT-B of the vasa recta moves it into the capillaries, and the erythrocyte UT-B moves it into and out of red blood cells to prevent cell disruption as the cells move through the blood vessels in the hyperosmolar portion of the renal medulla (“Theoretical effects of UTB urea transporters in the renal medullary microcirculation.” Zhang, W. and Edwards, A. Am. J. Physiol. 2003; 285(4, Pt 2): F731-F747.).

In the research on the isoforms of UT-A and UT-B, a few studies have reported their expression as either proteins, fragments of oligopeptides, or as RNA in portions of the gastrointestinal tract. UT-B is a protein with a molecular weight of approximately 40,000 which is glycosylated to produce a group of molecules with molecular weights between 45,000 and 65,000. The significance of the level of glycosylation is not currently known. UT-B mRNA has been found in the colon of rats (“Localization of the urea transporter UT-B protein in human and rat erythrocytes and tissues.” Timmer, R., Klein, J., Bagnasco, S., Doran, J., Verlander, J., Gunn, R., and Sands, J. Am. J. Physiol. (Cell Physiol.) 2001; 281: C1318-C1325) though human colonic tissue was not examined. UT-B has also been determined histologically to be present and the glycosylated protein in mouse erythrocytes, brain, kidney, bladder, spleen, and testes, and as the unglycosylated protein in esophagus, stomach, duodenum, colon, and rectum (“UT-B urea transporter is widely distributed in murine tissues and down-regulated by water deprivation in the bladder.” Lucien, N., Lasbennes, F., Roudier, N., Cartron, J., Bailly, P. J. Am. Soc Nephrol 2002; 13: F-P0035). One isoform of UT-A was found in rabbit colon as a 50,213 molecular weight protein (from amino acid analysis) with no data on whether it is glycosylated in its natural setting (“Urea transporter polypeptide.” Hediger, M. U.S. Pat. No. 5,441,875). One study, published only in abstract form, indicates that refractive light flux experiments suggest that a UT-A1 urea transporter is active as a facilitated, passive diffusion transporter in the mouse colon (“Expression of UT-A urea transporters in mouse colonic crypts.” Stewart, G., Fenton, R., Smith, C. J. Am. Soc. Nephrol. 2002; 13: F-P0043). This UT-A1 transporter was glycosylated to produce glycoproteins of about 34,000 molecular weight, 48,000 molecular weight, 75,000 molecular weight, and 100,000 molecular weight. From the data of Lifson, the data of Fordtran, and the data of Beck cited above, it was felt that these facilitated transporters were not efficient in allowing the passive movement of urea into or out of the colon.

Thus, current understanding of the gastrointestinal tract is that nitrogenous wastes move into the lumen of the intestine via passive diffusion with poor permeability of the intestinal mucosa to the wastes. Facilitated passive transport of urea has been described but has been shown under normal fasting and fed conditions to be of such a limited extent as to not interfere with the use of intraluminal urea as an unchanging osmotic agent in intestinal studies.

U.S. Pat. Nos. 5,679,717; 5,693,675; 5,618,530; 5,702,696; 5,607,669; 5,487,888 and 4,605,701 describe the ingestion of crosslinked alkylated amine polymers to remove bile salts and/or iron from a patient. However, these references teach removal of dietary iron before absorption or bile acids normally secreted into the bile by the liver. They do not teach or suggest activating transporters for metabolic waste.

U.S. Pat. No. 4,470,975 describes the elimination of water from the gastrointestinal (GI) tract by ingesting an insoluble, hydrophilic crosslinked polysaccharide which absorbs water from the gastrointestinal (GI) tract and is subsequently excreted. However, this reference does not teach or suggest removal of metabolic wastes.

Imondi, A. R. and Wolgemuth, R. L reported on their investigation of several insoluble resins, two polysaccharide preparations, various oxystarch preparations, and a polyacrylic acid resin as intestinal absorbents of nitrogenous wastes in uremic animals (“Gastrointestinal sorbents for the treatment of uremia. I. Lightly cross-linked carboxyvinyl polymer” in Ann. Nutr. Metab. 1981; 25: 311-319). The agents were delivered by gastric rather than intestinal administration. They note that the gastrically delivered oxystarch and the polyacrylic acid increased the fecal excretion of urea and total nitrogen to the same extent—about twice the amount excreted by rats fed cellulose. Ammonia, fluid, sodium, potassium, calcium, and magnesium were removed by the polyacrylic acid in amounts two to three times higher than the cellulose or oxystarch. The oxystarch caused diarrhea and colonic mucosal changes whereas the polyacrylic acid resin appeared to be tolerated except for the extreme removal of potassium, magnesium, and calcium. They found that polyacrylic acid resin as they were using it was not sufficient to remove enough urea through the gastrointestinal tract to have any impact on serum urea with either low or high protein intakes. They decided that the capacity of the polyacrylic acid resin for binding calcium was its most useful feature and patented its use for prevention of calcific renal stones through binding dietary calcium (U.S. Pat. No. 4,143,130). Although they did not note it, the gastric delivery of these agents caused them to be exposed to gastric acid followed by exposure to the hepatic bile, the pancreatic bicarbonate, and the pancreatic digestive enzymes. These exposures to strong acid, moderate base, and hydrolytic enzymes alter the chemical nature of the compounds used in their investigation and their effects on the gastrointestinal tract and its contents. They do not indicate any effects of the compounds other than the absorption or adsorption of compounds onto the polymers tested.

Japanese Patent Application Kokai No. H10-59851 (Application No. H8-256387) and Japanese Patent Application Kokai No. H10-130154 (Application No. H8-286446) disclose the administration into the stomach of alkali metal and alkaline earth salts of crosslinked polyacrylates dispersed into an oil emulsion to treat acute kidney failure for prolonging survival times. Their experiments look primarily at how long rats survive after total surgical nephrectomy. They consider the ability of the polymers they investigate to absorb physiologic saline, guanidine compounds, potassium, sodium, magnesium, and calcium. They do not examine effects on urea or creatinine. Since the polymer is introduced into the stomach, it is exposed to the stomach acid and upper small intestinal digestive compounds, just as is the case in the experiments reported by Imondi and Wolgemuth. They note the same removal of fluid and potassium and note that the calcium salt prolongs the rat survival time the longest, though they do not investigate why the agent with the lowest saline absorption of all the tested agents prolonged survival time the longest. They only consider the absorptive capabilities of the polymers without any consideration of how these substances are present in the intestine to be absorbed.

WO 02/040039 describes the in vivo use of water absorbent polymers to remove fluid from the intestinal tract and also describes removing metabolic waste. However, this reference teaches using functional groups on the polymer to facilitate waste removal and does not address activating metabolic waste transporters.

In all of the work on urea transporters to the present date, the nitrogenous wastes are understood to be merely facilitated in moving from a higher concentration in the bloodstream passively into the lower concentration in the gastrointestinal tract. No literature reports on possible transporters of creatinine or urates in the intestinal tract.

### SUMMARY OF THE INVENTION

The present invention has the advantage of concentrating the nitrogenous wastes in the intestinal tract to levels higher than those reached through passive diffusion. Furthermore, the present invention advantageously optimizes the removal of metabolic waste from the body by activating active transporters of nitrogenous metabolic waste. Having this activation be independent of forming covalent attachment of the agent to such metabolic waste products avoids the necessity of a complex and possibly lengthy reaction with the waste products.

In one aspect, the present invention is a method for stimulating active transporters of metabolic waste in the GI tract of a mammal, comprising the step of administering an effective amount of a concentrator activation agent to the intestinal tract of the mammal. The presence of these active transporters for urea and creatinine has not been previously known, and no method has been described to stimulate them.

In a second aspect, the present invention is a method for increasing the concentrations of metabolic waste in the GI tract of a mammal above simultaneous concentration in the bloodstream, comprising the step of administering an effective amount of a concentrator activation agent to the intestinal tract of the mammal. The ability to produce these higher intestinal luminal concentrations than simultaneous blood concentrations of nitrogenous wastes such as urea and creatinine has not been previously known, and the current art states that they should not be possible.

Surprisingly, it is believed that the use of the present invention stimulates active transporters of metabolic waste from the bloodstream into the GI tract, despite the fact that urea transporters have previously been thought to be passive uniporters and to generally not be involved in moving significant amounts of nitrogenous wastes into or out of the intestinal tract. Similarly, the presence of active transporters of creatinine into the intestinal tract has not been previously known and the surprising activation of these transporters by the agents of this invention has not been previously known. The present invention activates the metabolic waste transporters without the need for functional groups on the agents to covalently bind to the metabolic wastes.

Surprisingly, the use of the present invention produces concentrations of metabolic waste in portions of the intestinal tract that are higher than those in the bloodstream, despite the fact that urea has been previously thought to be moved into and out of the intestine by only passive uniporters which could not create a higher concentration of urea in the intestinal tract and which were thought to generally move relatively insignificant amounts of urea. Similarly, urates, creatinine, and other nitrogenous metabolic wastes were thought to move only through passive transport with very low permeability coefficients. The present invention concentrates the metabolic wastes in the intestine without the need for functional groups on the agents to covalently bind to the metabolic wastes.

### DETAILED DESCRIPTION OF THE INVENTION

Likewise, the subject invention involves directly delivering a non-systemic, non-toxic, non-digestible, concentrator activation agent to the intestinal tract of a host where it produces concentrations of metabolic wastes higher than those in the bloodstream. Although not wishing to be bound by theory, it is currently our belief that this concentration of metabolic wastes occurs through the stimulation of active transporters for the metabolic wastes which are located in portions of the gastrointestinal tract and are capable of moving urea, creatinine, and other metabolic wastes into the intestine against a concentration gradient (in greater quantities than passive diffusion across the intestinal membrane). The use of the concentrator activation agent allows the concentration of waste to be higher in the intestinal lumen than in the bloodstream. This allows significant excretion of metabolic wastes into the intestine and out of the body via the feces. The terms “concentrator activation agent” and “transporter activation agent” are used interchangeably throughout this application to mean the agent that is administered to a mammal in order to achieve the increase in concentration of metabolic waste.

Nitrogenous wastes are most appropriate for removal using the present invention. Examples of nitrogenous wastes include urea, uric acid, creatinine, and combinations thereof. These nitrogenous metabolic wastes are normally excreted through the urinary tract and minimal amounts of nitrogenous wastes have been measured to be excreted through the gastrointestinal tract. The present invention has been able to cause excretion of as much as 30% to 50% of the metabolically produced urea and creatinine through the feces.

In order to safely activate the metabolic waste transporters, the agent is directly delivered to the intestinal tract. The term “directly delivered” is intended to mean that the agent is not directly exposed to the stomach prior to delivery to the GI tract. One preferred means of directly delivering the agent to the GI tract is via oral administration of an enterically coated agent. The enteric coating protects the agent as it passes through the stomach such that the agent does not significantly degrade as a result of exposure to stomach acid. Moreover, the enteric coating prevents significant absorption or adsorption of nutrients or water from the stomach or upper small intestine. Upon reaching the intestinal tract, the enteric coating exposes or “releases” the agent where toxins or wastes are then expressed into the intestinal lumen and absorbed or adsorbed. The agent is subsequently excreted in the feces wherein the agent and the absorbed or adsorbed toxins or wastes are removed from the body. Other non-limiting examples of direct delivery of the agent include: introduction using an enema with large volume, a tube that is placed through the nose or mouth and empties directly into the desired portion of the intestine, a tube surgically implanted through the abdomen that empties into the intestine, and via intestinal lavage administration.

In a preferred embodiment, the transporter activation agent is a water absorbing polymer. Applicable polymers include polyelectrolyte and non-polyelectrolyte compounds. Polyelectrolyte polymers include, but are not limited to, carboxylate containing polymers such as polyacrylates, polyaspartates, polylactates, polyglucuronates, and the like as either homopolymers or copolymers, sulfonate containing polymers, and physiologically quaternary or cationic amine containing polymers such as polyallylamine or polyethyleneimine. Non-polyelectrolyte polymers, or non-ionic polymers, include such polymers as polyacrylamide gels, polyvinyl alcohol gels, and polyurethane gels. Preferred polymers include “super absorbent” acrylic polymers. The invention may include mixtures of other polymers in addition to the water absorbing polymers. Some polymers in this mixture may include finctional groups for selectively removing blood borne waste products e.g. urea, from the G.I. tract. One modality of this invention involves the use of multiple polymer components to remove water and a series of waste products. The subject polymers may be enterically coated such that they are protected from stomach acid but are exposed or “released” in the intestinal tract. Alternatively, the subject polymers may be administered through means, such as intestinal tubes, which allow placement directly into the desired portion of the intestine.

In another preferred embodiment of the invention, the transporter activation agent is a toxin absorbing/adsorbing agent. Applicable agents include activated charcoal, fullerene compounds, fulleroid compounds, and cyclodextrin compounds. One modality of this invention involves the use of multiple agents in mixtures to optimize the activation of transporters and the absorption/adsorption of uremic toxins. The subject agents and polymers may be enterically coated such that they are protected from the stomach and upper small intestine and released in the intestinal tract. Alternatively, the subject polymers and agents may be administered through means, such as intestinal tubes, which allow placement directly into the desired portion of the intestine.

The agents of the subject invention are generally easy to produce and many are commercially available.

The subject polymers include crosslinked polyacrylates which are water absorbent such as those prepared from α,β-ethylenically unsaturated monomers such as monocarboxylic acids, polycarboxylic acids, acrylamide and their derivatives, e.g. polymers having repeating units of acrylic acid, methacrylic acid, metal salts of acrylic acid, acrylamide, and acrylamide derivatives (such as 2-acrylamido-2-methylpropanesulfonic acid) along with various combinations of such repeating units as copolymers. Such derivatives include acrylic polymers which include hydrophilic grafts of polymers such as polyvinyl alcohol. Examples of suitable polymers and processes, including gel polymerization processes, for preparing such polymers are disclosed in U.S. Patent Nos. 3,997,484; 3,926,891; 3,935,099; 4,090,013; 4,093,776; 4,340,706; 4,446,261; 4,683,274; 4,459,396; 4,708,997; 4,076,663; 4,190,562; 4,286,082; 4,857,610; 4,985,518;

5,145,906; and 5,629,377, which are incorporated herein by reference. In addition, see Buchholz, F. L. and Graham, A. T., “Modem Superabsorbent Polymer Technology,” John Wiley & Sons (1998). Preferred polymers of the subject invention are polyelectrolytes. The degree of crosslinking can vary greatly depending upon the specific polymer material; however, in most applications the subject superabsorbent polymers are only lightly crosslinked, that is, the degree of crosslinking is such that the polymer can still absorb over 10 times its weight in physiological saline (i.e. 0.9% saline). For example, such polymers typically include less than about 0.2 mole percent crosslinking agent.

Different morphological forms of the polymers are possible. Polymers discussed in Buchholz, F. L. and Graham, A. T. Modem Superabsorbent Polymer Technology, John Wiley & Sons (1998) are generally irregularly shaped with sharp corners. Other morphological forms of crosslinked polyacrylates can be prepared by techniques discussed in EP 314825, U.S. Pat. No. 4833198, 4708997, WO 00/50096 and U.S. Pat. No. 1999-121329 incorporated herein by reference. These include several methods for preparing spherical bead forms and films. The bead forms, as prepared by methods similar to Example 1 of EP 314825 or Example 1 or Example 2 in WO 00/50096, are particularly advantageous for the present invention because the uptake of fluid and the swelling are more gradual. The irregularly shaped polymer reaches its maximum fluid absorption within 2 hours of placement into saline. Since the normal transit time through the stomach is 1.5 hours and the normal transit time through the small intestine is 1.5 hours, most of the fluid absorption of this polymer would occur in the small intestine. The bead form of the polymer swells to its maximum extent 10 hours after being exposed to saline. This allows the bead form of polymer to absorb more fluid in the distal small intestine and colon than occurs with the irregularly shaped polymer form. Absorbing more fluid in the distal portion of the intestine prevents interference with the normal intestinal absorption of nutrients and drugs while absorbing fluid that has a higher concentration of waste products. Swelling of the polymer in the colon also prevents feelings of fullness or bloating that may occur when the swelling occurs in the stomach.

Many of these polymers, regardless of the morphological form, are known for use as “super absorbents” and are commonly used in controlled release applications and personal hygiene products. Other agents of the present invention are commonly known as size-exclusion gels or water purification polymers. For the subject invention, food and/or pharmaceutical grades of materials are preferred. Although the alkali metal and alkaline metal salts of many of these polymers can be used (e.g. calcium, potassium, etc.); the sodium salt is particularly preferred.

Subject agents also include polysaccharides which may be used in the subject invention so long as such polysaccharides are directly administered to the intestinal tract and are not exposed to the stomach. For example, the polysaccharides described in U.S. Pat. No. 4,470,975 may be formulated as a tablet or provided within a capsule which is enterically coated and orally administered. Cyclodextrin molecules have been considered as oral agents for drug delivery, but have not been used for their absorptive ability or stimulatory ability (WO 2000018423 and “Biopharamceutical aspects of the tolbutamide-beta-cyclodextrin inclusion compound” Vila-Jato, J., Blanco, J., and Torres, J. Farmaco, Edizione Pratica 1988; 43: 37-45). In several embodiments of this invention, polysaccharide polymers are specifically avoided.

The quantity of transporter activation agent that is administered should be an amount that is effective to activate the metabolic waste transporters. Such an effective amount will depend upon the particular transporter activation agent selected. When the transporter activation agent is a water absorbent polymer, an effective amount of water absorbent polymer will generally have a wide range, e.g. from about 0.1 grams to about 50 grams per treatment but in some instances can be as high as about 100 grams per treatment. When the water absorbent polymer is a polyacrylate in particular, the effective amount of the polymer administered is typically between 1 gram and 50 grams. When the water absorbent polymer is a polysaccharide, the effective amount of the polymer administered is between 0.1 gram and 50 grams. When the transporter activation agent is a cyclodextrin type absorbent, the effective amount of the agent is between 0.1 gram and 200 grams. When the transporter activation agent is an activated charcoal of fullerene type agent, the effective dose is between 0.1 grams and 50 grams. When the transporter activation agent is a combination of these agents, the effective dose of each agent is within the range suggested for that agent.

In one embodiment of invention, the transporter activation agent is coated or encapsulated with an enteric material which prevents the release of agent in the stomach and delivers the agent directly to the intestine. The preferred delivery site is the distal jejunum, ileum, or colon. The enteric coatings used to encapsulate or coat the transporter activation agent ensure that the transporters in the intestinal tract are activated, because the transporter activation agent is still in its original form and has not degraded while passing through the stomach or upper small intestine. In contrast to previous art cited above, the present invention protects the transporter activation agent from exposure to gastric acid, thereby preserving the transporter activation performance. Moreover, by preventing the transporter activation agent from being exposed directly to the proximal small intestine, the present invention has less interference with normal absorption of nutrients and medications than the polymers mentioned in prior art.

Examples of such suitable enteric coatings include hydroxypropylmethyl cellulose, hydroxypropylmethyl cellulose phthalate, cellulose acetate phthalate, and sodium carboxyl methyl cellulose. Other suitable coatings are known in the art, e.g. polymers based on methacrylic acid and its derivatives, such as the EUDRAGIT copolymer systems, and are included within the scope of the present invention. The polymer may be provided within a capsule that is subsequently enterically coated. Multiple coatings may be utilized. When provided in bead or tablet form, the polymer may be directly coated. As previously mentioned, this invention includes other methods of delivering the subject polymers to the intestinal tract.

The result of the present invention is an increased quantity of metabolic waste exiting the body, as compared to using no transporter activation agents. Preferably, the level of metabolic waste removed using the present invention is increased by 5% and 60% of the total body store of the metabolic waste for the mammal. Preferably the amount of urea removed as a result of the agents activating urea transporters would be between 5% and 60% of the metabolically produced urea. Preferably the amount of uric acid removed as a result of the agents activating urate transporters would be between 5% and 60% of the metabolically produced urate. Preferably the amount of creatinine removed as a result of the agents activating creatinine transporters would be between 5% and 60% of the metabolically produced creatinine.

### EXAMPLES

### Example 1

Three Sprague-Dawley rats were fed rat chow as food. They were individually placed under isoflurane anesthesia to allow bilateral total nephrectomy. After nephrectomy, each rat received a measured amount of ¹⁴C urea intravenously and the abdominal incision was closed. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C urea in the plasma, the mean concentrations of ¹⁴C were 0.01 in the stomach, 0.87 in the duodenum, 1.56 in the proximal jejunum, 0.90 in the distal jejunum, 0.58 in the proximal ileum, 0.69 in the distal ileum, 0.19 in the cecum, 0.33 in the colon, and 0.80 in whole blood.

### Example 2

Three Sprague-Dawley rats were fed rat chow mixed with 50% by weight of a Sephadex G-100. They were individually placed under isoflurane anesthesia to allow bilateral total nephrectomy. After nephrectomy, each rat received a measured amount of ¹⁴C urea intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C urea in the plasma, the mean concentrations of ¹⁴C were 0.16 in the stomach, 1.14 in the duodenum, 1.24 in the proximal jejunum, 0.43 in the distal jejunum, 0.79 in the proximal ileum, 0.40 in the distal ileum, 0.11 in the cecum, 0.21 in the colon, and 0.46 in whole blood.

### Example 3

Three Sprague-Dawley rats were fed rat chow mixed with 5% of a lightly crosslinked polyacrylic acid that had been partially neutralized with sodium hydroxide. They were individually placed under isoflurane anesthesia to allow bilateral total nephrectomy. After nephrectomy, each rat received a measured amount of ¹⁴C urea intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C urea in the plasma, the mean concentrations of ¹⁴C were 0.61 in the stomach, 5.45 in the duodenum, 1.45 in the proximal jejunum, 2.58 in the distal jejunum, 1.87 in the proximal ileum, 2.37 in the distal ileum, 0.75 in the cecum, 0.86 in the colon, and 0.86 in whole blood.

### Example 4

Three Sprague-Dawley rats were fed rat chow as food. They were individually placed under isoflurane anesthesia to allow bilateral total nephrectomy. After nephrectomy, each rat received a measured amount of ¹⁴C creatinine intravenously and the abdominal incision was closed. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C creatinine in the plasma, the mean concentrations of ¹⁴C were 0.19 in the stomach, 1.10 in the duodenum, 1.11 in the proximal jejunum, 0.46 in the distal jejunum, 0.43 in the proximal ileum, 0.38 in the distal ileum, 0.12 in the cecum, 0.20 in the colon, and 0.77 in whole blood.

### Example 5

Three Sprague-Dawley rats were fed rat chow mixed with 50% by weight of a Sephadex G-100. They were individually placed under isoflurane anesthesia to allow bilateral total nephrectomy. After nephrectomy, each rat received a measured amount of ¹⁴C creatinine intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C creatinine in the plasma, the mean concentrations of ¹⁴C were 0.14 in the stomach, 1.40 in the duodenum, 1.90 in the proximal jejunum, 1.06 in the distal jejunum, 0.49 in the proximal ileum, 0.16 in the distal ileum, 0.06 in the cecum, 0.12 in the colon, and 0.27 in whole blood.

### Example 6

Three Sprague-Dawley rats were fed rat chow mixed with 5% of a lightly crosslinked polyacrylic acid that had been partially neutralized with sodium hydroxide. They were individually placed under isoflurane anesthesia to allow bilateral total nephrectomy. After nephrectomy, each rat received a measured amount of ¹⁴C creatinine intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C creatinine in the plasma, the mean concentrations of ¹⁴C were 0.65 in the stomach, 4.27 in the duodenum, 1.62 in the proximal jejunum, 2.40 in the distal jejunum, 1.32 in the proximal ileum, 1.11 in the distal ileum, 0.62 in the cecum, 0.84 in the colon, and 0.84 in whole blood.

### Example 7

Three Sprague-Dawley rats were fed rat chow as food. They were individually placed under isoflurane anesthesia to allow bilateral total nephrectomy. After nephrectomy, each rat received a measured amount of ¹⁴C uric acid intravenously and the abdominal incision was closed. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C uric acid in the plasma, the mean concentrations of ¹⁴C were 0.15 in the stomach, 0.76 in the duodenum, 0.44 in the proximal jejunum, 0.39 in the distal jejunum, 0.24 in the proximal ileum, 0.22 in the distal ileum, 0.07 in the cecum, 0.08 in the colon, and 0.57 in whole blood.

### Example 8

Three Sprague-Dawley rats were fed rat chow mixed with 50% by weight of a Sephadex G-100. They were individually placed under isoflurane anesthesia to allow bilateral total nephrectomy. After nephrectomy, each rat received a measured amount of ¹⁴C uric acid intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C uric acid in the plasma, the mean concentrations of ¹⁴C were 0.31 in the stomach, 0.62 in the duodenum, 0.45 in the proximal jejunum, 0.34 in the distal jejunum, 0.21 in the proximal ileum, 0.21 in the distal ileum, 0.07 in the cecum, 0.09 in the colon, and 0.55 in whole blood.

### Example 9

Three Sprague-Dawley rats were fed rat chow mixed with 5% of a lightly crosslinked polyacrytic acid that had been partially neutralized with sodium hydroxide. They were individually placed under isoflurane anesthesia to allow bilateral total nephrectomy. After nephrectomy, each rat received a measured amount of ¹⁴C uric acid intravenously and had abdominal closure. The rats remained under the isoflurane anesthesia for another 15 minutes and were then euthanized by exsanguination and isoflurane overdose. The blood was saved both as whole blood and as serum. The abdominal incisions were then opened to remove the stomach, the duodenum, the proximal jejunum, the distal jejunum, the proximal ileum, the distal ileum, the cecum, and the colon along with their respective contents. These samples were weighed, solubilized, and counted for ¹⁴C. Expressed as a decimal fraction of the concentration of ¹⁴C uric acid in the plasma, the mean concentrations of ¹⁴C were 0.28 in the stomach, 0.61 in the duodenum, 0.31 in the proximal jejunum, 0.49 in the distal jejunum, 0.17 in the proximal ileum, 0.27 in the distal ileum, 0.07 in the cecum, 0.09 in the colon, and 0.60 in whole blood.

TABLE 1
Tabular Data from Examples 1 to 9.

| | | Stomach | Duodenum | Jejunum-1 | Jejunum-2 | Ileum-1 | Ileum-2 | Cecum | Colon | Whole Blood |
|---|---|---|---|---|---|---|---|---|---|---|
| 14C Urea | Rodent Chow | 0.01 | 0.87 | 1.56 | 0.90 | 0.58 | 0.69 | 0.19 | 0.33 | 0.80 |
| 14C Urea | 50% Sephadex | 0.16 | 1.14 | 1.24 | 0.43 | 0.79 | 0.40 | 0.11 | 0.21 | 0.46 |
| | G-100 | | | | | | | | | |
| 14C Urea | 5% CLP | 0.61 | 5.45 | 1.45 | 2.58 | 1.87 | 2.37 | 0.75 | 0.86 | 0.86 |
| 14C Creatinine | Rodent Chow | 0.19 | 1.10 | 1.11 | 0.46 | 0.43 | 0.38 | 0.12 | 0.20 | 0.77 |
| 14C Creatinine | 50% Sephadex | 0.14 | 1.40 | 1.90 | 1.06 | 0.49 | 0.16 | 0.06 | 0.12 | 0.27 |
| | G-100 | | | | | | | | | |
| 14C Creatinine | 5% CLP | 0.65 | 4.27 | 1.62 | 2.40 | 1.32 | 1.11 | 0.62 | 0.84 | 0.84 |
| 14C Uric Acid | Rodent Chow | 0.15 | 0.76 | 0.44 | 0.39 | 0.24 | 0.22 | 0.07 | 0.08 | 0.57 |
| 14C Uric Acid | 50% Sephadex | 0.31 | 0.62 | 0.45 | 0.34 | 0.21 | 0.21 | 0.07 | 0.09 | 0.55 |
| | G-100 | | | | | | | | | |
| 14 C Uric Acid | 5% CLP | 0.28 | 0.61 | 0.31 | 0.49 | 0.17 | 0.27 | 0.07 | 0.09 | 0.60 |

Note:
The numbers in Table I represent a ratio of the organ concentration to the plasma concentration. Numbers above 1.0 indicate either active transport into the lumen or binding of the compound by some intraluminal substance. Similarly, increases in the numbers over those with only rodent chow indicate either binding of the compound by the agent mixed with the food or stimulation of secretion of the compound.

### Example 10

Four patients being treated with hemodialysis for End Stage Renal Disease were followed on their regular dialysis routine to determine the amount of urea generated between their dialysis sessions. The patients were then continued on their routine hemodialysis and additionally placed on 10 gram per day of enteric coated partial sodium salt of lightly crosslinked polyacrylic acid (“CLP”). The polymer absorbed and removed from the body approximately 0.55 liter of fluid per day. In the first patient, the CLP caused the removal of 473 mg of urea per day whereas passive diffusion of urea from the bloodstream into the feces to saturate 0.55 liter of fluid could have only removed a maximum of 167 mg of urea per day. In the second patient, the CLP caused the removal of 2190 mg of urea per day while a maximum of only 380 mg of urea could have been removed by passive diffusion of 0.55 liter of fluid. In the third patient, CLP caused the removal of 1276 mg of urea per day while passive diffusion of 0.55 liter of fluid could have only removed 294 mg of urea. In the fourth patient, CLP caused the removal of 1097 mg of urea per day while passive diffusion of 0.55 liter of fluid could have only removed a maximum of 340 mg of urea during the day.

### Example 11

Dry CLP was placed into an aqueous solution of urea and allowed to maximally absorb fluid. The swollen CLP was placed into a large amount of deionized water and allowed to equilibrate. The urea absorbed into the CLP from the first solution quickly moved into the deionized water.

What is claimed is:

1. A method for activating metabolic waste transporters in the intestinal tract of a mammal, comprising the step of:
administering an effective amount of a concentrator activation agent directly to the intestinal tract of the mammal.

2. The method according to claim 1 wherein the metabolic waste transporters are active transporters for nitrogenous wastes.

3. The method according to claim 2 wherein the nitrogenous wastes are selected from the group consisting of urea, uric acid, creatinine and combinations thereof.

4. The method according to claim 1 wherein the concentrator activation agent is a water absorbent polymer.

5. The method according to claim 4 wherein the water absorbent polymer is a polyacrylate.

6. The method according to claim 5 wherein the effective amount of the polymer administered is between 1 gram and 50 grams.

7. The method according to claim 4 wherein the water absorbent polymer is a polysaccharide.

8. The method according to claim 7 wherein the effective amount of the polymer administered is between 0.1 gram and 50 grams.

9. The method according to claim 4 wherein the water absorbent polymer is a polycarboxylic acid alkali metal salt.

10. The method according to claim 9 wherein the polycarboxylic acid alkali metal salt is a polycarboxylic acid sodium salt.

11. The method according to claim 10 wherein the polycarboxylic acid sodium salt is selected from the group consisting of sodium salts of polyacrylic acid, polyglutamic acid, polylactic acid, polyaspartic acid, polyglucuronic acid, or polysaccharides containing carboxylic acid units such as glucuronic acid units.

12. The method according to claim 1 wherein the water absorbent polymer is coated with an enteric coating.

13. The method according to claim 1 wherein activating the transporters results in an increased level of metabolic wastes in the intestinal tract, and wherein the method further comprises the step of removing the metabolic waste from the intestinal tract.

14. The method according to claim 13 wherein the increased level of metabolic waste in the intestinal tract is between 5% and 60% of the total body store of the metabolic waste for the mammal.

15. The method according to claim 13 wherein the increased level of metabolic waste in the intestinal tract is between 5% and 60% of the metabolically produced waste for the mammal on a daily basis.

16. The method according to claim 1 wherein the concentrator activation agent is an absorbent/adsorbent chosen from activated charcoal, fullerenes, fulleroids, and cyclodextrins or combinations of these agents.

17. The method according to claim 1 wherein the concentrator activation agent is a combination of two or more agents selected from the group consisting of a water absorbent polymer, activated charcoal, fullerenes, fulleroids, and cyclodextrins.

18. A method for removing metabolic wastes from a mammal through the intestinal tract, comprising the step of:
administering directly to the intestinal tract of the mammal a concentrator activation agent in an amount effective to concentrate metabolic wastes in the intestinal tract above a level that would be reached by passive diffusion alone.

19. The method according to claim 18 wherein the metabolic wastes are nitrogenous metabolic wastes.

20. The method according to claim 19 wherein the nitrogenous wastes are selected from the group consisting of urea, uric acid, creatinine and combinations thereof.

21. The method according to claim 18 wherein the concentrator activation agent is a water absorbent polymer.

22. The method according to claim 21 wherein the water absorbent polymer is a polyacrylate.

23. The method according to claim 22 wherein the effective amount of the polymer administered is between 1 gram and 50 grams.

24. The method according to claim 21 wherein the water absorbent polymer is a polysaccharide.

25. The method according to claim 22 wherein the effective amount of the polymer administered is between 0.1 gram and 50 grams.

26. The method according to claim 21 wherein the water absorbent polymer is a polycarboxylic acid alkali metal salt.

27. The method according to claim 26 wherein the polycarboxylic acid alkali metal salt is a polycarboxylic acid sodium salt.

28. The method according to claim 27 wherein the polycarboxylic acid sodium salt is selected from the group consisting of sodium salts of polyacrylic acid, polyglutamic acid, polylactic acid, polyaspartic acid, polyglucuronic acid, or polysaccharides containing carboxylic acid units such as glucuronic acid units.

29. The method according to claim 18 wherein the water absorbent polymer is coated with an enteric coating.

30. The method according to claim 18 wherein activating the transporters results in an increased level of metabolic wastes in the intestinal tract, and wherein the method further comprises the step of removing the metabolic waste from the intestinal tract.

31. The method according to claim 30 wherein the increased level of metabolic waste in the intestinal tract is between 5% and 60% of the total body store of the metabolic waste for the mammal.

32. The method according to claim 30 wherein the increased level of metabolic waste in the intestinal tract is between 5% and 60% of the metabolically produced waste for the mammal on a daily basis.

33. The method according to claim 18 wherein the concentrator activation agent is chosen from activated charcoal, fullerenes, fulleroids, or cyclodextrins.

34. The method according to claim 18 wherein the concentrator activation agent is a combination of two or more agents selected from the group consisting of a water absorbent polymer, activated charcoal, fullerenes, fulleroids, and cyclodextrins.
